(19) United States
(12) Patent Application Publication
(10) Pub. No.: US 2022/0031311 A1
(43) Pub. Date: Feb. 3, 2022

(54) MEDICAL SUTURE NEEDLE

(71) Applicant: MANI, INC., Utsunomiya-shi (JP)

(72) Inventor: Kazuhiko Matsutani, Utsunomiya-shi (JP)

(73) Assignee: MANI, INC., Utsunomiya-shi (JP)

(21) Appl. No.: 17/387,732

(22) Filed: Jul. 28, 2021

(30) Foreign Application Priority Data
Jul. 30, 2020 (JP) 2020-129098

(51) Int. Cl. A61B 17/06

(52) U.S. Cl. CPC A61B 17/06066

(57) ABSTRACT

The purpose of the present invention is to provide a medical suture needle with improved strength against repeated bending while ensuring visibility. The medical suture needle A including a needle tip part with a needle tip for piercing tissue, a base part for attaching a suture thread thereto, and a body part between the needle tip part and the base part, in which the body part includes at least one shallower groove 5 in a direction from the needle tip part to the body part, and the shallower groove 5 is defined by two opposing gentle slopes 6.

### TECHNICAL FIELD

This invention relates to a medical suture needle having higher visibility of an area to be grasped using a needle holder and a piercing direction when performing surgery, and having higher resistance to repeated bending.

### BACKGROUND ART

Medical suture needles contain a needle tip part for pierceing tissue, a base part for attaching a suture thread thereto, and a body part between the needle tip part and base part. The medical suture needle is grasped using a needle holder. Stitching surgeries are performed using the medical suture needle via the needle holder.

When the needle tip or base part of the medical suture needle is grasped using the needle holder, the medical suture needle may be damaged or piercing ability thereof may be impaired, resulting that operability thereof may decrease, strength thereof against bending may be reduced, and the suture thread may detach, making stitching surgery impossible. It is necessary to securely grasp the body part between the needle tip part and the base part.

In particular, in order to ensure the grasping of the body part using the needle holder to stabilize the surgery, a medical suture needle 50 with grooves 54 in a body part 53 between a needle tip part 51 and a base part 52 is conventionally provided, as shown in FIGS. 4A and 4B. In this medical suture needle 50, a cross-section of the body part 53 has a square shape, and two grooves 54 are provided in the longitudinal direction of the body part 53, and a protruding part 55 is provided between the grooves 54.

The medical suture needle 50 configured as described above can be firmly grasped using the needle holder, and a force of a practitioner can be reliably transmitted thereto. Furthermore, by matching an interval between the grooves 54, a shape of the groove 54, and a shape of the protruding part 55 with an interval between convex parts on a gripping surface of the needle holder, a shape of the convex part thereon and a shape of a concave part thereon, respectively, rotation of the medical suture needle 50 can be prevent during the surgery.

On the other hand, when the medical suture needle is used to suture tissue, a bending force is applied to the suture needle at the point where it is grasped using the needle holder. For this reason, the medical suture needle needs to have strong enough to withstand repeated bending.

Although the medical suture needle 50 with the grooves 54 in the longitudinal direction of the body part 53 as described above stabilizes the grasping of the needle holder, microcracks may occur in the protruding parts as the number of repetitive bending increases, which cause a problem of inferior durability. However, this problem can be solved by using a medical suture needle 57 without grooves on a body part 56, as shown in FIGS. 5A and 5B.

### SUMMARY OF INVENTION

### Technical Problem

Although the medical suture needle 57 can exhibit higher durability against repeated bending, boundaries among the body part 56, a needle tip part 58, and a base part 59 are invisible because the body part 56 has flat surfaces. In other words, there is a problem that the range of the body part 56 is invisible.

In particular, there are, in some users, problems that it is difficult to determine which portion of the body part should be grasped using the needle holder, and that the direction of movement of the medical suture needle during the stitching surgery is unclear. Among these users, there is a persistent demand for suture needles with grooves on the body.

Therefore, there are real needs to develop medical suture needles having both visibility and durability against the repeated bending.

The purpose of the present invention is to provide a medical suture needle with improved strength against the repeated bending while ensuring visibility.

### Solution to Problem

In order to solve the above problems, the inventor analyzed conventional medical suture needles with the grooves on the body part and developed a medical suture needle that can satisfy the aforementioned needs.

Therefore, visibility for the practitioner is needed to the medical suture needles with the grooves on the body. In other words, the visibility relates to the checks of the range of the body part that can be grasped using the needle holder and the direction of movement (piercing direction). For this reason, the inventor studied whether or not a groove composed of two opposing slopes can be visible regardless of the angle between the two slopes. Also, the durability of the suture needle with the grooves on the body part against the repeated bending was studied.

First, the inventor investigated the shape of the groove 54 on the body part 53 of the conventional medical suture needle 50 shown in FIGS. 4A and 4B, and also investigated its durability against the repeated bending. In the case of the medical suture needle 50, the angle between the two opposing slopes was in the range of 90 to 70 degrees, although most of them were 80 degrees, and the number of grooves 54 was two to four. The protruding parts existed among these grooves, and the protruding part has an angle equal to the angle of the groove.

For this purpose, a test piece of suture needle with grooves in which the angle between two opposing slopes is 80 degrees was manufactured and subjected to a 90-degree repeated bending test, as described below. The test piece was manufactured by pressing SUS302 wire with a diameter of 0.6 mm to form a drum body part having a drum cross-sectional shape, a square body part having a square cross-sectional shape, and a long body part having a rectangular cross-sectional shape and to form two grooves with an angle of 80 degrees between the two opposing slopes. An angle of a top of a protruding part between the two grooves was 80 degrees.

As a result of the 90-degree repeated bending test of the test piece described above, the drum body part broke after two bends, the square body part broke after 1.8 bends, and the long body part broke after 1.2 bends. As a result of the condition check of each of the broken test pieces, micro-cracks were found at the top of the protruding parts. The micro-cracks were caused by the concentration of stress due to the repeated bending. It is assumed that the breaking was caused by these microcracks.

Based on the above results and the fact that the body part without grooves has sufficient bending strength, in order to ensure the durability against the repeated bending, it is effective to increase the angle between one of the slopes and the surface of the body part, and the angle of the top of the protruding part between the grooves by increasing the angle between the two slopes constituting the groove. That is, it was expected that it would be possible to improve the bending strength of the medical suture needle due to shallower grooves defined by two opposing gentle slopes on the body part.

In addition, it was found that the range of the body part that can be grasped using the needle holder and the direction of movement (piercing direction) can be sufficiently checked by using the medical suture needle with the grooves on the body part.

Based on the result, the inventor came up with a medical suture needle with grooves on a body part that are visible to the practitioner and have sufficient the durability against the repeated bending. Thus, a typical medical suture needle of the present invention includes a needle tip part with a needle tip for piercing tissue, a base part for attaching a suture thread thereto, and a body part between the needle tip part and the base part, in which the body part includes at least one shallower groove in a direction from the needle tip part to the body part, and the shallower groove is defined by two opposing gentle slopes.

### Advantageous Effect of the Invention

In the medical suture needle of the present invention (hereinafter simply referred to as “suture needle”), the groove on the body part is defined by two opposing gentle slopes, and the angle between the gentle slope and the surface of the body part, or the angle of the top of the protruding part between the two grooves is larger. As a result, a stress concentrated when the bending is applied can be reduced, and the strength against the repeated bending can be increased.

Since the groove is defined by the gentle slopes, light irradiated on the surface of the body part is reflected in different directions from the surface of the body part and from the gentle slopes of the groove, resulting that the groove can be sufficiently visible. Therefore, the practitioner can visually check an area grasped using the needle holder and a direction of movement of the suture needle.

### BRIEF DESCRIPTION OF THE DRAWINGS

FIG. 1A shows a schematic perspective view of a suture needle, according to the present embodiment.

FIG. 1B shows a cross-sectional view of a body part of the suture needle shown in FIG. 1A, according to the present embodiment.

FIG. 2A illustrates a cross-sectional view of the body part of a test piece used in a repeated bending test.

FIG. 2B illustrates a cross-sectional view of the body part of a test piece used in a repeated bending test.

FIG. 2C illustrates a cross-sectional view of the body part of a test piece used in a repeated bending test.

FIG. 3 illustrates the repeated bending test.

FIG. 4A shows a schematic perspective view of a conventional medical suture needle.

FIG. 4B shows a cross-sectional view of a body part of the conventional medical suture needle shown in FIG. 4A.

FIG. 5A shows a schematic perspective view of a conventional medical suture needle.

FIG. 5B shows a cross-sectional view of a body part of the conventional medical suture needle shown in FIG. 5A.

### DESCRIPTION OF EMBODIMENTS

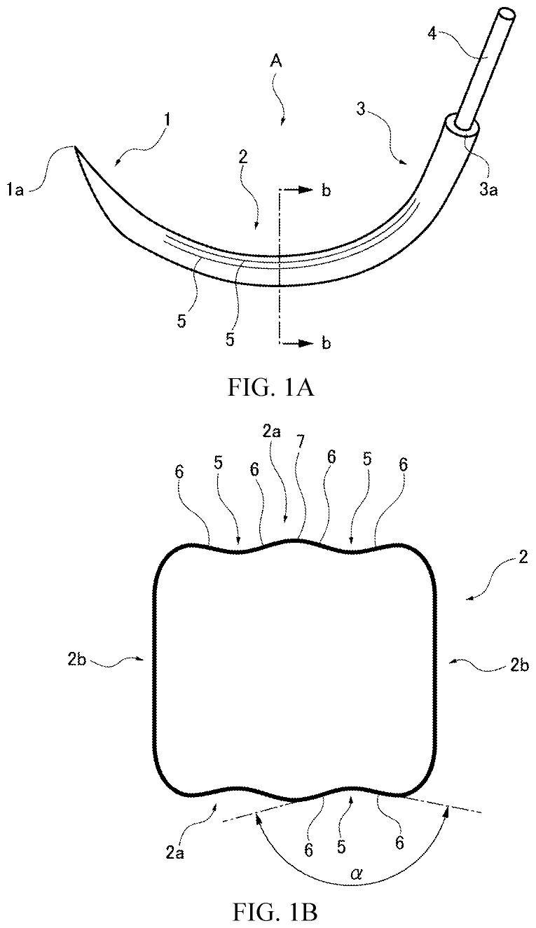

Embodiments of a suture needle in accordance with the present invention will be described using FIGS. 1A to 3. A suture needle A in this embodiment has a needle tip part 1, a body part 2 connected with the needle tip 1, and a base part 3 connected with the body part 2, in which each of opposite sides 2a of the body part 2 has two grooves 5. The suture needle A in this embodiment is configured as a curved needle having a curved shape from the needle tip part 1 to the base part 3. A plurality of types of the curved shape and thickness of the suture needle A are standardized in advance, and most appropriate standard is selected for an area to be sutured.

A material of the suture needle A may be steel wire, martensitic stainless steel, and austenitic stainless steel, but not limited thereto. In the case of the steel wire or the martensitic stainless steel, hardening by heat treatment can be expected, but it is difficult to eliminate occurrence of rust. In the case of the austenitic stainless steel, although the hardening by heat treatment cannot be expected, there is no risk of the occurrence of rust. Thus, it is preferable to use the austenitic stainless steel in consideration of the occurrence of rust.

In this embodiment, a material processed by cold drawing of a wire made of the austenitic stainless steel with a preset surface reduction to achieve higher hardness and then stretching the austenite structure into fibers to improve bending strength is used.

The needle tip part 1 has a needle tip 1a for piercing tissue at a tip thereof, and thickness of the needle tip 1a increases from the tip to the body part 2. In general, length of the needle tip part 1 is about 6 to 12 times thickness of the body part 2, but any lengths are also available to meet the purpose of the suture needle. The length of the needle tip part 1 is configured so that it increases as the thickness of the body part 2 increases.

The body part 2 is a part that is grasped using a needle holder, and thickness thereof is set within the range of 0.025 mm to 2.00 mm. Practitioners select the suture needle A with the most suitable thickness according to affected areas to be sutured.

A cross-sectional shape of the body part 2 may be a triangle, a drum with two opposite sides configured as flat surfaces, or a square with four sides configured as flat surfaces, but not particularly limited thereto. Also, in this embodiment, the suture needle A is a curved needle, but it may be a straight needle.

The body part 2 has two grooves 5 and a protruding part 7 between the grooves 5 on each of opposite sides 2a of the body part 2. The groove 5 and protruding part 7 are extended in the longitudinal direction of the suture needle A. The groove 5 is defined by two opposing gentle slopes 6 with an angle of 120 degrees or more. The groove 5 is preferably a visible groove. In other words, the angle between the two opposing gentle slopes is preferably 120 degrees or more, and the upper limit of the angle is preferably an angle that can be recognized as a groove. The upper limit of the angle is about 170 degrees.

In a cross-sectional view of the body part 2, a maximum angle between tangent lines on the parts constituting the groove 5 is 120 degrees or more. In other words, when a vertical line is set at the bottom of the groove 5, an angle between the vertical line and one gentle slope 6 does not need to be equal to an angle between the vertical line and the other gentle slope 6, but the maximum angle between the tangent lines of gentle slope 6 is preferably 120 degrees or more.

Since the groove 5 is defined by the two opposing gentle slopes 6, the angle of the top of the protruding part 7 between the two grooves 5 is larger, resulting in improving the durability against the repeated bending applied to the suture needle A.

Depth of the groove 5 is 20% or less of the thickness of the body part 2. That is, if the depth of the groove 5 is larger than 20% of the thickness of the body part 2, it may adversely affect the durability of the body part 2 against the bending. When the depth of the groove 5 is smaller than 20% of the thickness of the body part 2, the durability is not reduced. Regardless of the depth, the groove 5 is essential in order to prevent the visibility to the practitioner from decreasing.

For example, if width of a surface 2a of the body part 2 is 1 mm, effective width thereof is 80%, and the angle between the opposing gentle slopes 6 is 120 degrees, the depth of the groove 5 is about 0.11 mm, which is 11% of the thickness of the body part 2. When a single groove 5 is on the surface 2a, the depth of the groove 5 is about 0.22 mm, which is 22% of the depth. Therefore, it is preferable that the angle between the two gentle slopes 6 constituting the groove 5 is larger than 120 degrees such as 140 degrees, and the depth of the groove 5 is smaller than 20% of the thickness of the body part 2.

The base part 3 is for attaching the suture thread 4 thereto, which may be a suture needle having a through hole and spring property corresponding to a function of suture needles or a suture needle having a stopper hole on an end surface 3a of the base part 3. In this embodiment, the base part 3 has a circular cross-section and is configured as a suture needle with the hole on the end surface 3a for attaching the suture thread 4 thereto.

A method for producing the suture needle A with the above shape may include: pressing a material with a circular cross-section to form a square and at least one groove 5; pressing two opposite sides of a material with a circular cross-section to form surfaces 2a and 2b and at least one groove; and grinding or pressing a material to form surfaces 2a and 2b, and then irradiating a laser beam to form at least one groove 5 on the surface 2a.

### Example

The inventor conducted comparative experiments on the relationship between the angle of the groove and the durability against the repeated bending.

Suture needles with grooves having an angle in accordance with the present invention (120 degrees and 140 degrees), suture needles without grooves, suture needles with grooves having the same angle as conventional suture needles (80 degrees), and suture needles with grooves having an angle (100 degrees) for comparison were tested. The results of the tests are briefly described below.

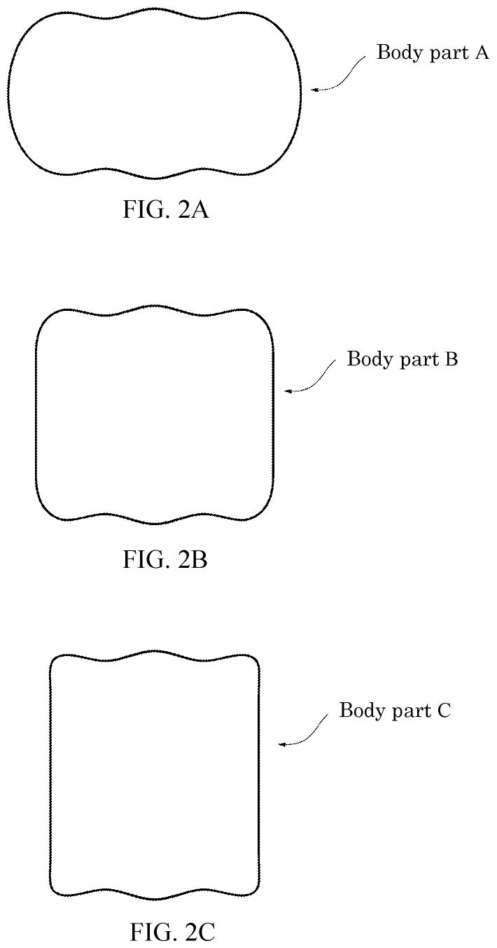

First, circular materials were pressed to form a drum-shaped body part (body part A) having a drum cross-sectional shape shown in FIG. 2A, a square body part (body part B) having a square cross-sectional shape shown in FIG. 2B, and a long body part (body part C) having a rectangular cross-sectional shape shown in FIG. 2C.

The suture needles with grooves having any one of angles of 80 degrees (A80, B80 and C80 which are typical groove angles in conventional suture needles described above), 100 degrees (A100, B100 and C100), 120 degrees (A120, B120 and C120), and 140 degrees (A140, B140 and C140) defined by two opposing gentle slopes on each body part were prepared. Furthermore, the suture needles without grooves on the body (A180, B180 and C180) were prepared. Five test pieces were prepared for each of the aforementioned conditions.

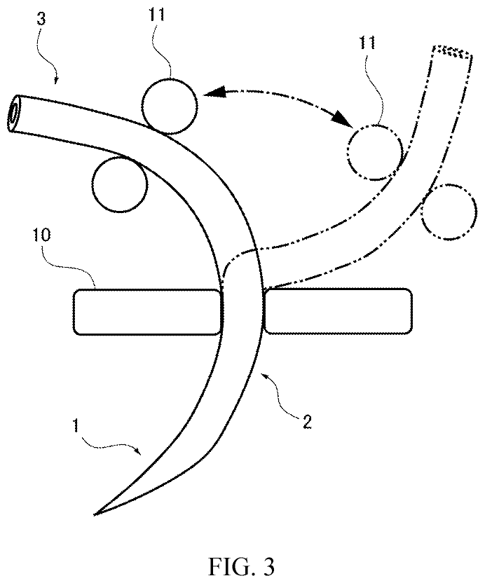

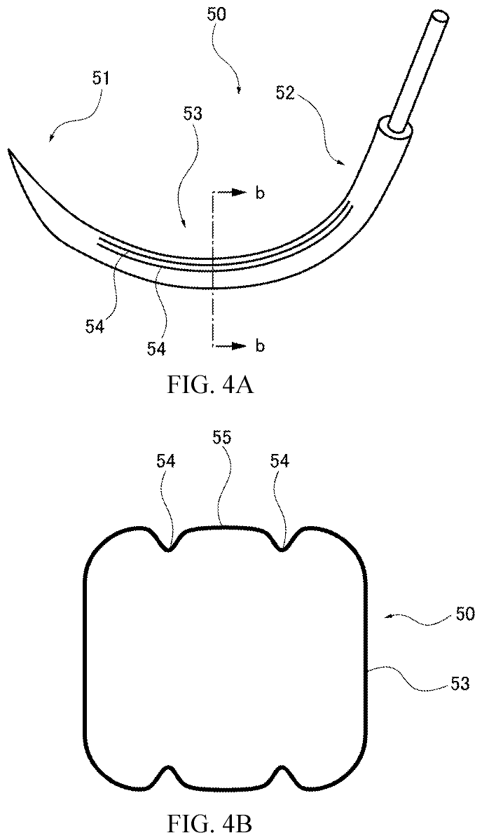

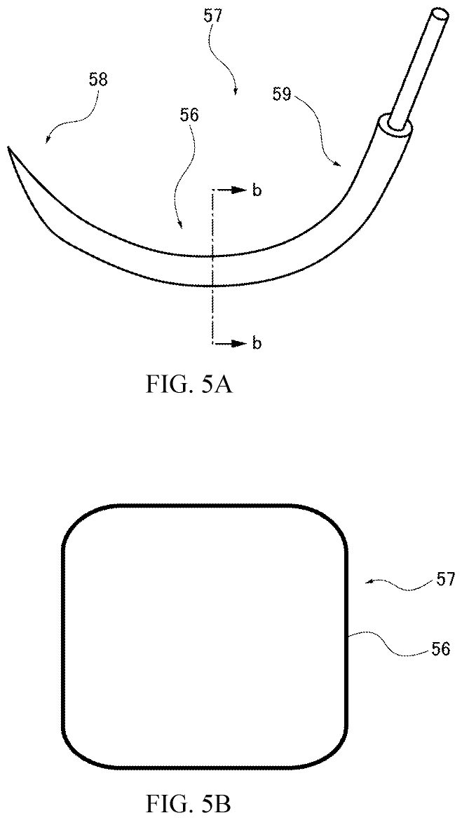

As shown in FIG. 3, a vise 10 was used to hold the tip of the needle 1 on the body part 2 of each test piece, and a force-applying means 11 was used to apply a force to the base part 3 to bend the piece about 90 degrees repeatedly. The number of bending times was measured until the needle broke, and the average value for each condition was used to make an evaluation. Suture needles with an average number of the repeated bending leading to breakage of 3 or more were judged to have passed the test.

The results of the above tests are as follows: A80 was 2.0 times, B80 was 1.6 times, and C80 was 1.2 times; A100 was 3.8 times, B100 was 2.4 times, and C100 was 2.0 times; A120 was 8.8 times, B120 was 5.2 times, and C120 was 4.8 times; A140 was 9.2 times, B140 was 5.3 times, C140 was 5.2 times; and A180 was 9.4 times, B180 was 6.0 times, and C180 was 5.4 times.

Based on the results of the above tests, it was found that the suture needle with the grooves having the angle of 120 degrees or 140 degrees defined by the two opposing gentle slopes on the body part has sufficient durability against the repeated bending. In addition, since the groove has the gentle slope on the surface of the body part, the light irradiated on the body is not totally reflected, and the practitioner can see the existence of the grooves sufficiently.

In the aforementioned example, two grooves 5 were formed on each of the opposite sites 2a of the body part 2, but this is not limited to two grooves. Three grooves or even one groove can be acceptable. The groove 5 allow doctors to check an area to be grasped using the needle holder and a suturing direction. For this reason, it is not necessary to have at least one groove on each surfaces of the body part 21. It may have at least one groove on a single surface of the body part 21.

### INDUSTRIAL APPLICABILITY

As described above, the suture needle A of the present invention allows the practitioner to visualize the range of the area that can be grasped using the needle holder and direction of motion, and exhibits sufficient durability against the repeated bending. For these reasons, it can be used regardless of the external shape or cross-sectional shape of the suture needle.

### DESCRIPTION OF THE REFERENCE NUMERAL

A Suture needle

1 Needle tip part

1a Needle tip

2 Body part

2a, 2b Surface

3 Body part

3a End surface

4 Suture thread

5 Groove

6 Gentle slope

7 Protruding part

10 Vise

11 Force-applying means

What is claimed is:

1. A medical suture needle comprising a needle tip part with a needle tip for piercing tissue, a base part for attaching a suture thread thereto, and a body part between the needle tip part and the base part,
wherein the body part comprises at least one shallower groove in a direction from the needle tip part to the body part, and the shallower groove is defined by two opposing gentle slopes.

2. The medical suture needle according to claim 1, wherein an angle between the gentle slopes constituting the groove is 120 degrees or more.

3. The medical suture needle according to claim 1, wherein the groove comprises depth of 20% or less of thickness of the body part.

4. The medical suture needle according to claim 1, wherein in a cross-sectional view of the body part, a maximum angle between tangent lines on parts constituting the groove 5 is 120 degrees or more.
